SEQUENCE LISTING (132 sequences)

SEQ ID NO: 1 - DNA, 51 bp, Artificial Sequence
  Description of Artificial Sequence Synthetic oligonucleotide
  Description of Combined DNA/RNA Molecule Synthetic oligonucleotide
  modified_base (25)..(26): a, c, t, g, unknown or other
  modified_base (28)..(29): a, c, t, g, unknown or other
  modified_base (31)..(31): a, c, t, g, unknown or other
  modified_base (44)..(51): a, c, t, g, unknown or other
aacuacacgacgcucuuccgaucunnwnnwnatgccatgccgtnnnnnnnn

SEQ ID NO: 2 - DNA, 51 bp, Artificial Sequence
  Description of Artificial Sequence Synthetic oligonucleotide
  Description of Combined DNA/RNA Molecule Synthetic oligonucleotide
  modified_base (25)..(26): a, c, t, g, unknown or other
  modified_base (28)..(29): a, c, t, g, unknown or other
  modified_base (31)..(31): a, c, t, g, unknown or other
  modified_base (44)..(51): a, c, t, g, unknown or other
aacuacacgacgcucuuccgaucunnwnnwnaccatccaacgannnnnnnn

SEQ ID NO: 3 - DNA, 51 bp, Artificial Sequence
  Description of Artificial Sequence Synthetic oligonucleotide
  Description of Combined DNA/RNA Molecule Synthetic oligonucleotide
  modified_base (25)..(26): a, c, t, g, unknown or other
  modified_base (28)..(29): a, c, t, g, unknown or other
  modified_base (31)..(31): a, c, t, g, unknown or other
  modified_base (44)..(51): a, c, t, g, unknown or other
aacuacacgacgcucuuccgaucunnwnnwngtggcctactacnnnnnnnn

SEQ ID NO: 4 - DNA, 51 bp, Artificial Sequence
  Description of Artificial Sequence Synthetic oligonucleotide
  Description of Combined DNA/RNA Molecule Synthetic oligonucleotide
  modified_base (25)..(26): a, c, t, g, unknown or other
  modified_base (28)..(29): a, c, t, g, unknown or other
  modified_base (31)..(31): a, c, t, g, unknown or other
  modified_base (44)..(51): a, c, t, g, unknown or other
aacuacacgacgcucuuccgaucunnwnnwnggagttgaggtgnnnnnnnn

SEQ ID NO: 5 - DNA, 51 bp, Artificial Sequence
  Description of Artificial Sequence Synthetic oligonucleotide
  Description of Combined DNA/RNA Molecule Synthetic oligonucleotide
  modified_base (25)..(26): a, c, t, g, unknown or other
  modified_base (28)..(29): a, c, t, g, unknown or other
  modified_base (31)..(31): a, c, t, g, unknown or other
  modified_base (44)..(51): a, c, t, g, unknown or other
aacuacacgacgcucuuccgaucunnwnnwnattgcaagcaacnnnnnnnn

SEQ ID NO: 6 - DNA, 51 bp, Artificial Sequence
  Description of Artificial Sequence Synthetic oligonucleotide
  Description of Combined DNA/RNA Molecule Synthetic oligonucleotide
  modified_base (25)..(26): a, c, t, g, unknown or other
  modified_base (28)..(29): a, c, t, g, unknown or other
  modified_base (31)..(31): a, c, t, g, unknown or other
  modified_base (44)..(51): a, c, t, g, unknown or other
aacuacacgacgcucuuccgaucunnwnnwntacaaccgagtannnnnnnn

SEQ ID NO: 7 - DNA, 51 bp, Artificial Sequence
  Description of Artificial Sequence Synthetic oligonucleotide
  Description of Combined DNA/RNA Molecule Synthetic oligonucleotide
  modified_base (25)..(26): a, c, t, g, unknown or other
  modified_base (28)..(29): a, c, t, g, unknown or other
  modified_base (31)..(31): a, c, t, g, unknown or other
  modified_base (44)..(51): a, c, t, g, unknown or other
aacuacacgacgcucuuccgaucunnwnnwntgcgttctagcgnnnnnnnn

SEQ ID NO: 8 - DNA, 51 bp, Artificial Sequence
  Description of Artificial Sequence Synthetic oligonucleotide
  Description of Combined DNA/RNA Molecule Synthetic oligonucleotide
  modified_base (25)..(26): a, c, t, g, unknown or other
  modified_base (28)..(29): a, c, t, g, unknown or other
  modified_base (31)..(31): a, c, t, g, unknown or other
  modified_base (44)..(51): a, c, t, g, unknown or other
aacuacacgacgcucuuccgaucunnwnnwntgttcctctcacnnnnnnnn

SEQ ID NO: 9 - DNA, 51 bp, Artificial Sequence
  Description of Artificial Sequence Synthetic oligonucleotide
  Description of Combined DNA/RNA Molecule Synthetic oligonucleotide
  modified_base (25)..(26): a, c, t, g, unknown or other
  modified_base (28)..(29): a, c, t, g, unknown or other
  modified_base (31)..(31): a, c, t, g, unknown or other
  modified_base (44)..(51): a, c, t, g, unknown or other
aacuacacgacgcucuuccgaucunnwnnwntgcagtcctcgannnnnnnn

SEQ ID NO: 10 - DNA, 51 bp, Artificial Sequence
  Description of Artificial Sequence Synthetic oligonucleotide
  Description of Combined DNA/RNA Molecule Synthetic oligonucleotide
  modified_base (25)..(26): a, c, t, g, unknown or other
  modified_base (28)..(29): a, c, t, g, unknown or other
  modified_base (31)..(31): a, c, t, g, unknown or other
  modified_base (44)..(51): a, c, t, g, unknown or other
aacuacacgacgcucuuccgaucunnwnnwncgatacactgccnnnnnnnn

SEQ ID NO: 11 - DNA, 51 bp, Artificial Sequence
  Description of Artificial Sequence Synthetic oligonucleotide
  Description of Combined DNA/RNA Molecule Synthetic oligonucleotide
  modified_base (25)..(26): a, c, t, g, unknown or other
  modified_base (28)..(29): a, c, t, g, unknown or other
  modified_base (31)..(31): a, c, t, g, unknown or other
  modified_base (44)..(51): a, c, t, g, unknown or other
aacuacacgacgcucuuccgaucunnwnnwntctgcgagtctgnnnnnnnn

SEQ ID NO: 12 - DNA, 51 bp, Artificial Sequence
  Description of Artificial Sequence Synthetic oligonucleotide
  Description of Combined DNA/RNA Molecule Synthetic oligonucleotide
  modified_base (25)..(26): a, c, t, g, unknown or other
  modified_base (28)..(29): a, c, t, g, unknown or other
  modified_base (31)..(31): a, c, t, g, unknown or other
  modified_base (44)..(51): a, c, t, g, unknown or other
aacuacacgacgcucuuccgaucunnwnnwntcttgcggagtcnnnnnnnn

SEQ ID NO: 13 - DNA, 51 bp, Artificial Sequence
  Description of Artificial Sequence Synthetic oligonucleotide
  Description of Combined DNA/RNA Molecule Synthetic oligonucleotide
  modified_base (25)..(26): a, c, t, g, unknown or other
  modified_base (28)..(29): a, c, t, g, unknown or other
  modified_base (31)..(31): a, c, t, g, unknown or other
  modified_base (44)..(51): a, c, t, g, unknown or other
aacuacacgacgcucuuccgaucunnwnnwnaggcttacgtgtnnnnnnnn

SEQ ID NO: 14 - DNA, 51 bp, Artificial Sequence
  Description of Artificial Sequence Synthetic oligonucleotide
  Description of Combined DNA/RNA Molecule Synthetic oligonucleotide
  modified_base (25)..(26): a, c, t, g, unknown or other
  modified_base (28)..(29): a, c, t, g, unknown or other
  modified_base (31)..(31): a, c, t, g, unknown or other
  modified_base (44)..(51): a, c, t, g, unknown or other
aacuacacgacgcucuuccgaucunnwnnwntcacgagtcacannnnnnnn

SEQ ID NO: 15 - DNA, 51 bp, Artificial Sequence
  Description of Artificial Sequence Synthetic oligonucleotide
  Description of Combined DNA/RNA Molecule Synthetic oligonucleotide
  modified_base (25)..(26): a, c, t, g, unknown or other
  modified_base (28)..(29): a, c, t, g, unknown or other
  modified_base (31)..(31): a, c, t, g, unknown or other
  modified_base (44)..(51): a, c, t, g, unknown or other
aacuacacgacgcucuuccgaucunnwnnwnagccggagagtannnnnnnn

SEQ ID NO: 16 - DNA, 51 bp, Artificial Sequence
  Description of Artificial Sequence Synthetic oligonucleotide
  Description of Combined DNA/RNA Molecule Synthetic oligonucleotide
  modified_base (25)..(26): a, c, t, g, unknown or other
  modified_base (28)..(29): a, c, t, g, unknown or other
  modified_base (31)..(31): a, c, t, g, unknown or other
  modified_base (44)..(51): a, c, t, g, unknown or other
aacuacacgacgcucuuccgaucunnwnnwnatggaaggtggcnnnnnnnn

SEQ ID NO: 17 - DNA, 41 bp, Artificial Sequence
  Description of Artificial Sequence Synthetic oligonucleotide
  modified_base (13)..(13): a, c, t, g, unknown or other
  modified_base (15)..(16): a, c, t, g, unknown or other
  modified_base (18)..(19): a, c, t, g, unknown or other
acggcatggcatnwnnwnnagatcggaagagcgtcgtgtag

SEQ ID NO: 18 - DNA, 41 bp, Artificial Sequence
  Description of Artificial Sequence Synthetic oligonucleotide
  modified_base (13)..(13): a, c, t, g, unknown or other
  modified_base (15)..(16): a, c, t, g, unknown or other
  modified_base (18)..(19): a, c, t, g, unknown or other
tcgttggatggtnwnnwnnagatcggaagagcgtcgtgtag

SEQ ID NO: 19 - DNA, 41 bp, Artificial Sequence
  Description of Artificial Sequence Synthetic oligonucleotide
  modified_base (13)..(13): a, c, t, g, unknown or other
  modified_base (15)..(16): a, c, t, g, unknown or other
  modified_base (18)..(19): a, c, t, g, unknown or other
gtagtaggccacnwnnwnnagatcggaagagcgtcgtgtag

SEQ ID NO: 20 - DNA, 41 bp, Artificial Sequence
  Description of Artificial Sequence Synthetic oligonucleotide
  modified_base (13)..(13): a, c, t, g, unknown or other
  modified_base (15)..(16): a, c, t, g, unknown or other
  modified_base (18)..(19): a, c, t, g, unknown or other
cacctcaactccnwnnwnnagatcggaagagcgtcgtgtag

SEQ ID NO: 21 - DNA, 41 bp, Artificial Sequence
  Description of Artificial Sequence Synthetic oligonucleotide
  modified_base (13)..(13): a, c, t, g, unknown or other
  modified_base (15)..(16): a, c, t, g, unknown or other
  modified_base (18)..(19): a, c, t, g, unknown or other
gttgcttgcaatnwnnwnnagatcggaagagcgtcgtgtag

SEQ ID NO: 22 - DNA, 41 bp, Artificial Sequence
  Description of Artificial Sequence Synthetic oligonucleotide
  modified_base (13)..(13): a, c, t, g, unknown or other
  modified_base (15)..(16): a, c, t, g, unknown or other
  modified_base (18)..(19): a, c, t, g, unknown or other
tactcggttgtanwnnwnnagatcggaagagcgtcgtgtag

SEQ ID NO: 23 - DNA, 41 bp, Artificial Sequence
  Description of Artificial Sequence Synthetic oligonucleotide
  modified_base (13)..(13): a, c, t, g, unknown or other
  modified_base (15)..(16): a, c, t, g, unknown or other
  modified_base (18)..(19): a, c, t, g, unknown or other
cgctagaacgcanwnnwnnagatcggaagagcgtcgtgtag

SEQ ID NO: 24 - DNA, 41 bp, Artificial Sequence
  Description of Artificial Sequence Synthetic oligonucleotide
  modified_base (13)..(13): a, c, t, g, unknown or other
  modified_base (15)..(16): a, c, t, g, unknown or other
  modified_base (18)..(19): a, c, t, g, unknown or other
gtgagaggaacanwnnwnnagatcggaagagcgtcgtgtag

SEQ ID NO: 25 - DNA, 41 bp, Artificial Sequence
  Description of Artificial Sequence Synthetic oligonucleotide
  modified_base (13)..(13): a, c, t, g, unknown or other
  modified_base (15)..(16): a, c, t, g, unknown or other
  modified_base (18)..(19): a, c, t, g, unknown or other
tcgaggactgcanwnnwnnagatcggaagagcgtcgtgtag

SEQ ID NO: 26 - DNA, 41 bp, Artificial Sequence
  Description of Artificial Sequence Synthetic oligonucleotide
  modified_base (13)..(13): a, c, t, g, unknown or other
  modified_base (15)..(16): a, c, t, g, unknown or other
  modified_base (18)..(19): a, c, t, g, unknown or other
ggcagtgtatcgnwnnwnnagatcggaagagcgtcgtgtag

SEQ ID NO: 27 - DNA, 41 bp, Artificial Sequence
  Description of Artificial Sequence Synthetic oligonucleotide
  modified_base (13)..(13): a, c, t, g, unknown or other
  modified_base (15)..(16): a, c, t, g, unknown or other
  modified_base (18)..(19): a, c, t, g, unknown or other
cagactcgcaganwnnwnnagatcggaagagcgtcgtgtag

SEQ ID NO: 28 - DNA, 41 bp, Artificial Sequence
  Description of Artificial Sequence Synthetic oligonucleotide
  modified_base (13)..(13): a, c, t, g, unknown or other
  modified_base (15)..(16): a, c, t, g, unknown or other
  modified_base (18)..(19): a, c, t, g, unknown or other
gactccgcaaganwnnwnnagatcggaagagcgtcgtgtag

SEQ ID NO: 29 - DNA, 41 bp, Artificial Sequence
  Description of Artificial Sequence Synthetic oligonucleotide
  modified_base (13)..(13): a, c, t, g, unknown or other
  modified_base (15)..(16): a, c, t, g, unknown or other
  modified_base (18)..(19): a, c, t, g, unknown or other
acacgtaagcctnwnnwnnagatcggaagagcgtcgtgtag

SEQ ID NO: 30 - DNA, 41 bp, Artificial Sequence
  Description of Artificial Sequence Synthetic oligonucleotide
  modified_base (13)..(13): a, c, t, g, unknown or other
  modified_base (15)..(16): a, c, t, g, unknown or other
  modified_base (18)..(19): a, c, t, g, unknown or other
tgtgactcgtganwnnwnnagatcggaagagcgtcgtgtag

SEQ ID NO: 31 - DNA, 41 bp, Artificial Sequence
  Description of Artificial Sequence Synthetic oligonucleotide
  modified_base (13)..(13): a, c, t, g, unknown or other
  modified_base (15)..(16): a, c, t, g, unknown or other
  modified_base (18)..(19): a, c, t, g, unknown or other
tactctccggctnwnnwnnagatcggaagagcgtcgtgtag

SEQ ID NO: 32 - DNA, 41 bp, Artificial Sequence
  Description of Artificial Sequence Synthetic oligonucleotide
  modified_base (13)..(13): a, c, t, g, unknown or other
  modified_base (15)..(16): a, c, t, g, unknown or other
  modified_base (18)..(19): a, c, t, g, unknown or other
gccaccttccatnwnnwnnagatcggaagagcgtcgtgtag

SEQ ID NO: 33 - DNA, 40 bp, Artificial Sequence
  Description of Artificial Sequence Synthetic primer
ggatctcgacgctctccctctccttactcatggtcggatc

SEQ ID NO: 34 - DNA, 41 bp, Artificial Sequence
  Description of Artificial Sequence Synthetic primer
ggatctcgacgctctcccttcctttgcaggactgtcaagca

SEQ ID NO: 35 - DNA, 40 bp, Artificial Sequence
  Description of Artificial Sequence Synthetic primer
ggatctcgacgctctccctgtcctttctgtaggctggatg

SEQ ID NO: 36 - DNA, 40 bp, Artificial Sequence
  Description of Artificial Sequence Synthetic primer
ggatctcgacgctctccctttttgagtttgcagactttcc

SEQ ID NO: 37 - DNA, 41 bp, Artificial Sequence
  Description of Artificial Sequence Synthetic primer
ggatctcgacgctctccctggattgcagattgggccttggg

SEQ ID NO: 38 - DNA, 39 bp, Artificial Sequence
  Description of Artificial Sequence Synthetic primer
ggatctcgacgctctccctggatgtcacgttctcaaagc

SEQ ID NO: 39 - DNA, 41 bp, Artificial Sequence
  Description of Artificial Sequence Synthetic primer
ggatctcgacgctctccctagcgctactagaaacatgatag

SEQ ID NO: 40 - DNA, 42 bp, Artificial Sequence
  Description of Artificial Sequence Synthetic primer
ggatctcgacgctctcccttgttgactttttagttttacttt

SEQ ID NO: 41 - DNA, 40 bp, Artificial Sequence
  Description of Artificial Sequence Synthetic primer
ggatctcgacgctctccctaggtcagtggatcccctctcc

SEQ ID NO: 42 - DNA, 41 bp, Artificial Sequence
  Description of Artificial Sequence Synthetic primer
ggatctcgacgctctcccttgaagaagatgtggaaaagtcc

SEQ ID NO: 43 - DNA, 40 bp, Artificial Sequence
  Description of Artificial Sequence Synthetic primer
ggatctcgacgctctccctctgtcctcatgtattggtctc

SEQ ID NO: 44 - DNA, 43 bp, Artificial Sequence
  Description of Artificial Sequence Synthetic primer
ggatctcgacgctctccctcagaaaacaagtggttatagatgg

SEQ ID NO: 45 - DNA, 41 bp, Artificial Sequence
  Description of Artificial Sequence Synthetic primer
ggatctcgacgctctcccttccacctttctccagctaattc

SEQ ID NO: 46 - DNA, 40 bp, Artificial Sequence
  Description of Artificial Sequence Synthetic primer
ggatctcgacgctctccctgctccatgcagatactgatct

SEQ ID NO: 47 - DNA, 39 bp, Artificial Sequence
  Description of Artificial Sequence Synthetic primer
ggatctcgacgctctccctgacacaacacaaaatagccg

SEQ ID NO: 48 - DNA, 41 bp, Artificial Sequence
  Description of Artificial Sequence Synthetic primer
ggatctcgacgctctccctactccaggataatacacatcac

SEQ ID NO: 49 - DNA, 40 bp, Artificial Sequence
  Description of Artificial Sequence Synthetic primer
ggatctcgacgctctcccttgcaggctcaccccaatgcag

SEQ ID NO: 50 - DNA, 45 bp, Artificial Sequence
  Description of Artificial Sequence Synthetic primer
ggatctcgacgctctccctagctgctgaaaatgtaactttgtatc

SEQ ID NO: 51 - DNA, 40 bp, Artificial Sequence
  Description of Artificial Sequence Synthetic primer
ggatctcgacgctctccctcaaaatggatccagacaactg

SEQ ID NO: 52 - DNA, 42 bp, Artificial Sequence
  Description of Artificial Sequence Synthetic primer
ggatctcgacgctctccctgcttgctctgataggaaaatgag

SEQ ID NO: 53 - DNA, 39 bp, Artificial Sequence
  Description of Artificial Sequence Synthetic primer
ggatctcgacgctctccctagcaagtatgatgagcaagc

SEQ ID NO: 54 - DNA, 40 bp, Artificial Sequence
  Description of Artificial Sequence Synthetic primer
ggatctcgacgctctccctatgctctgagaaaggcattag

SEQ ID NO: 55 - DNA, 40 bp, Artificial Sequence
  Description of Artificial Sequence Synthetic primer
ggatctcgacgctctccctgattttgcagaaaacagatct

SEQ ID NO: 56 - DNA, 40 bp, Artificial Sequence
  Description of Artificial Sequence Synthetic primer
ggatctcgacgctctccctgccttctagaacagtagacac

SEQ ID NO: 57 - DNA, 44 bp, Artificial Sequence
  Description of Artificial Sequence Synthetic primer
ggatctcgacgctctccctaaatgattctgaattagctgtatcg

SEQ ID NO: 58 - DNA, 44 bp, Artificial Sequence
  Description of Artificial Sequence Synthetic primer
ggatctcgacgctctcccttttattataaggcctgctgaaaatg

SEQ ID NO: 59 - DNA, 39 bp, Artificial Sequence
  Description of Artificial Sequence Synthetic primer
ggatctcgacgctctccctaaagaaagccctccccagtc

SEQ ID NO: 60 - DNA, 43 bp, Artificial Sequence
  Description of Artificial Sequence Synthetic primer
ggatctcgacgctctccctcctacaggaagcaagtagtaattg

SEQ ID NO: 61 - DNA, 40 bp, Artificial Sequence
  Description of Artificial Sequence Synthetic primer
ggatctcgacgctctccctgttgaagtcctcgttgtcttg

SEQ ID NO: 62 - DNA, 39 bp, Artificial Sequence
  Description of Artificial Sequence Synthetic primer
ggatctcgacgctctccctttgcactccctcaggtagtc

SEQ ID NO: 63 - DNA, 41 bp, Artificial Sequence
  Description of Artificial Sequence Synthetic primer
ggatctcgacgctctccctcagttaacgtcttccttctctc

SEQ ID NO: 64 - DNA, 39 bp, Artificial Sequence
  Description of Artificial Sequence Synthetic primer
ggatctcgacgctctccctggacccccacacagcaaagc

SEQ ID NO: 65 - DNA, 40 bp, Artificial Sequence
  Description of Artificial Sequence Synthetic primer
ggatctcgacgctctccctttacacccagtggagaagctc

SEQ ID NO: 66 - DNA, 39 bp, Artificial Sequence
  Description of Artificial Sequence Synthetic primer
ggatctcgacgctctccctcccaccagaccatgagaggc

SEQ ID NO: 67 - DNA, 39 bp, Artificial Sequence
  Description of Artificial Sequence Synthetic primer
ggatctcgacgctctccctaacgtactggtgaaaacacc

SEQ ID NO: 68 - DNA, 39 bp, Artificial Sequence
  Description of Artificial Sequence Synthetic primer
ggatctcgacgctctccctcaggaaaatgctggctgacc

SEQ ID NO: 69 - DNA, 40 bp, Artificial Sequence
  Description of Artificial Sequence Synthetic primer
ggatctcgacgctctccctgtggacaacccccacgtgtgc

SEQ ID NO: 70 - DNA, 40 bp, Artificial Sequence
  Description of Artificial Sequence Synthetic primer
ggatctcgacgctctccctctgggagccaatattgtcttt

SEQ ID NO: 71 - DNA, 42 bp, Artificial Sequence
  Description of Artificial Sequence Synthetic primer
ggatctcgacgctctccctacattattgccaacatgacttac

SEQ ID NO: 72 - DNA, 39 bp, Artificial Sequence
  Description of Artificial Sequence Synthetic primer
ggatctcgacgctctccctaatatcccccggcttgtgag

SEQ ID NO: 73 - DNA, 43 bp, Artificial Sequence
  Description of Artificial Sequence Synthetic primer
ggatctcgacgctctccctgtgtccaccgtgatctggctgctc

SEQ ID NO: 74 - DNA, 40 bp, Artificial Sequence
  Description of Artificial Sequence Synthetic primer
ggatctcgacgctctcccttgaggacgtacactgcctttc

SEQ ID NO: 75 - DNA, 41 bp, Artificial Sequence
  Description of Artificial Sequence Synthetic primer
ggatctcgacgctctcccttgacattgcatacattcgaaag

SEQ ID NO: 76 - DNA, 39 bp, Artificial Sequence
  Description of Artificial Sequence Synthetic primer
ggatctcgacgctctcccttttcagttcaatgcatgctg

SEQ ID NO: 77 - DNA, 41 bp, Artificial Sequence
  Description of Artificial Sequence Synthetic primer
ggatctcgacgctctccctggaaaatgacaaagaacagctc

SEQ ID NO: 78 - DNA, 40 bp, Artificial Sequence
  Description of Artificial Sequence Synthetic primer
ggatctcgacgctctccctccattttagcacttacctgtg

SEQ ID NO: 79 - DNA, 40 bp, Artificial Sequence
  Description of Artificial Sequence Synthetic primer
ggatctcgacgctctcccttagtaggggaagatcatctgc

SEQ ID NO: 80 - DNA, 40 bp, Artificial Sequence
  Description of Artificial Sequence Synthetic primer
ggatctcgacgctctccctgccggagcagctgcgcaacag

SEQ ID NO: 81 - DNA, 39 bp, Artificial Sequence
  Description of Artificial Sequence Synthetic primer
ggatctcgacgctctccctgggcatcactgtaaaccttg

SEQ ID NO: 82 - DNA, 39 bp, Artificial Sequence
  Description of Artificial Sequence Synthetic primer
ggatctcgacgctctcccttcactgacagccctctggac

SEQ ID NO: 83 - DNA, 60 bp, Artificial Sequence
  Description of Artificial Sequence Synthetic primer
ttgactggagttcagacgtgtgctcttccgatcttccttactcatggtcggatcacaaag

SEQ ID NO: 84 - DNA, 60 bp, Artificial Sequence
  Description of Artificial Sequence Synthetic primer
ttgactggagttcagacgtgtgctcttccgatctcctttgcaggactgtcaagcagagaa

SEQ ID NO: 85 - DNA, 60 bp, Artificial Sequence
  Description of Artificial Sequence Synthetic primer
ttgactggagttcagacgtgtgctcttccgatctgtaggctggatgaaaaattcacagtc

SEQ ID NO: 86 - DNA, 60 bp, Artificial Sequence
  Description of Artificial Sequence Synthetic primer
ttgactggagttcagacgtgtgctcttccgatctttttgagtttgcagactttccaaagc

SEQ ID NO: 87 - DNA, 54 bp, Artificial Sequence
  Description of Artificial Sequence Synthetic primer
ttgactggagttcagacgtgtgctcttccgatctggctgagtcctggcgctgtg

SEQ ID NO: 88 - DNA, 54 bp, Artificial Sequence
  Description of Artificial Sequence Synthetic primer
ttgactggagttcagacgtgtgctcttccgatctcgttctcaaagcagtggatc

SEQ ID NO: 89 - DNA, 59 bp, Artificial Sequence
  Description of Artificial Sequence Synthetic primer
ttgactggagttcagacgtgtgctcttccgatctctactagaaacatgatagaggtgac

SEQ ID NO: 90 - DNA, 60 bp, Artificial Sequence
  Description of Artificial Sequence Synthetic primer
ttgactggagttcagacgtgtgctcttccgatcttgttattaatatgagtattgttaacc

SEQ ID NO: 91 - DNA, 54 bp, Artificial Sequence
  Description of Artificial Sequence Synthetic primer
ttgactggagttcagacgtgtgctcttccgatcttggcctacctggtcgccatg

SEQ ID NO: 92 - DNA, 55 bp, Artificial Sequence
  Description of Artificial Sequence Synthetic primer
ttgactggagttcagacgtgtgctcttccgatctagatgtggaaaagtcccaatg

SEQ ID NO: 93 - DNA, 60 bp, Artificial Sequence
  Description of Artificial Sequence Synthetic primer
ttgactggagttcagacgtgtgctcttccgatctctgtcctcatgtattggtctctcatg

SEQ ID NO: 94 - DNA, 60 bp, Artificial Sequence
  Description of Artificial Sequence Synthetic primer
ttgactggagttcagacgtgtgctcttccgatctaaaacaagtggttatagatggtgaaa

SEQ ID NO: 95 - DNA, 56 bp, Artificial Sequence
  Description of Artificial Sequence Synthetic primer
ttgactggagttcagacgtgtgctcttccgatcttttctccagctaattcatctgg

SEQ ID NO: 96 - DNA, 56 bp, Artificial Sequence
  Description of Artificial Sequence Synthetic primer
ttgactggagttcagacgtgtgctcttccgatctgccatcgctgtagaacgcacca

SEQ ID NO: 97 - DNA, 58 bp, Artificial Sequence
  Description of Artificial Sequence Synthetic primer
ttgactggagttcagacgtgtgctcttccgatctacacaacacaaaatagccgtataa

SEQ ID NO: 98 - DNA, 57 bp, Artificial Sequence
  Description of Artificial Sequence Synthetic primer
ttgactggagttcagacgtgtgctcttccgatctcatcacagtaaataacactctgg

SEQ ID NO: 99 - DNA, 58 bp, Artificial Sequence
  Description of Artificial Sequence Synthetic primer
ttgactggagttcagacgtgtgctcttccgatctcaggctcaccccaatgcagcgaac

SEQ ID NO: 100 - DNA, 58 bp, Artificial Sequence
  Description of Artificial Sequence Synthetic primer
ttgactggagttcagacgtgtgctcttccgatctatgtaactttgtatcctgttcctc

SEQ ID NO: 101 - DNA, 55 bp, Artificial Sequence
  Description of Artificial Sequence Synthetic primer
ttgactggagttcagacgtgtgctcttccgatctacaactgttcaaactgatggg

SEQ ID NO: 102 - DNA, 60 bp, Artificial Sequence
  Description of Artificial Sequence Synthetic primer
ttgactggagttcagacgtgtgctcttccgatcttgctctgataggaaaatgagatctac

SEQ ID NO: 103 - DNA, 55 bp, Artificial Sequence
  Description of Artificial Sequence Synthetic primer
ttgactggagttcagacgtgtgctcttccgatctatgatgagcaagctttctcac

SEQ ID NO: 104 - DNA, 58 bp, Artificial Sequence
  Description of Artificial Sequence Synthetic primer
ttgactggagttcagacgtgtgctcttccgatctgcattagaaagcctgtagttttac

SEQ ID NO: 105 - DNA, 57 bp, Artificial Sequence
  Description of Artificial Sequence Synthetic primer
ttgactggagttcagacgtgtgctcttccgatctaacagatctgtatttatttcagt

SEQ ID NO: 106 - DNA, 60 bp, Artificial Sequence
  Description of Artificial Sequence Synthetic primer
ttgactggagttcagacgtgtgctcttccgatctgccttctagaacagtagacacaaaac

SEQ ID NO: 107 - DNA, 58 bp, Artificial Sequence
  Description of Artificial Sequence Synthetic primer
ttgactggagttcagacgtgtgctcttccgatcttctgaattagctgtatcgtcaagg

SEQ ID NO: 108 - DNA, 54 bp, Artificial Sequence
  Description of Artificial Sequence Synthetic primer
ttgactggagttcagacgtgtgctcttccgatctggcctgctgaaaatgactga

SEQ ID NO: 109 - DNA, 54 bp, Artificial Sequence
  Description of Artificial Sequence Synthetic primer
ttgactggagttcagacgtgtgctcttccgatcttccccagtcctcatgtactg

SEQ ID NO: 110 - DNA, 59 bp, Artificial Sequence
  Description of Artificial Sequence Synthetic primer
ttgactggagttcagacgtgtgctcttccgatctaggaagcaagtagtaattgatggag

SEQ ID NO: 111 - DNA, 54 bp, Artificial Sequence
  Description of Artificial Sequence Synthetic primer
ttgactggagttcagacgtgtgctcttccgatctgtcctcgttgtcttgttggc

SEQ ID NO: 112 - DNA, 58 bp, Artificial Sequence
  Description of Artificial Sequence Synthetic primer
ttgactggagttcagacgtgtgctcttccgatctgcactccctcaggtagtccaggag

SEQ ID NO: 113 - DNA, 60 bp, Artificial Sequence
  Description of Artificial Sequence Synthetic primer
ttgactggagttcagacgtgtgctcttccgatctgttaacgtcttccttctctctctgtc

SEQ ID NO: 114 - DNA, 55 bp, Artificial Sequence
  Description of Artificial Sequence Synthetic primer
ttgactggagttcagacgtgtgctcttccgatctcccacacagcaaagcagaaac

SEQ ID NO: 115 - DNA, 54 bp, Artificial Sequence
  Description of Artificial Sequence Synthetic primer
ttgactggagttcagacgtgtgctcttccgatctggagaagctcccaaccaagc

SEQ ID NO: 116 - DNA, 55 bp, Artificial Sequence
  Description of Artificial Sequence Synthetic primer
ttgactggagttcagacgtgtgctcttccgatctcagaccatgagaggccctgcg

SEQ ID NO: 117 - DNA, 54 bp, Artificial Sequence
  Description of Artificial Sequence Synthetic primer
ttgactggagttcagacgtgtgctcttccgatctggtgaaaacaccgcagcatg

SEQ ID NO: 118 - DNA, 57 bp, Artificial Sequence
  Description of Artificial Sequence Synthetic primer
ttgactggagttcagacgtgtgctcttccgatctgaaaatgctggctgacctaaagc

SEQ ID NO: 119 - DNA, 54 bp, Artificial Sequence
  Description of Artificial Sequence Synthetic primer
ttgactggagttcagacgtgtgctcttccgatctcccacgtgtgccgcctgctg

SEQ ID NO: 120 - DNA, 56 bp, Artificial Sequence
  Description of Artificial Sequence Synthetic primer
ttgactggagttcagacgtgtgctcttccgatctagccaatattgtctttgtgttc

SEQ ID NO: 121 - DNA, 60 bp, Artificial Sequence
  Description of Artificial Sequence Synthetic primer
ttgactggagttcagacgtgtgctcttccgatctattgccaacatgacttacttgatccc

SEQ ID NO: 122 - DNA, 60 bp, Artificial Sequence
  Description of Artificial Sequence Synthetic primer
ttgactggagttcagacgtgtgctcttccgatctaatatcccccggcttgtgagtggatg

SEQ ID NO: 123 - DNA, 56 bp, Artificial Sequence
  Description of Artificial Sequence Synthetic primer
ttgactggagttcagacgtgtgctcttccgatctcgtgatctggctgctcgcaacg

SEQ ID NO: 124 - DNA, 59 bp, Artificial Sequence
  Description of Artificial Sequence Synthetic primer
ttgactggagttcagacgtgtgctcttccgatctgaggacgtacactgcctttcgacac

SEQ ID NO: 125 - DNA, 57 bp, Artificial Sequence
  Description of Artificial Sequence Synthetic primer
ttgactggagttcagacgtgtgctcttccgatctagccttagataaaactgagcaag

SEQ ID NO: 126 - DNA, 57 bp, Artificial Sequence
  Description of Artificial Sequence Synthetic primer
ttgactggagttcagacgtgtgctcttccgatctcatgctgtttaattgtgtggaag

SEQ ID NO: 127 - DNA, 59 bp, Artificial Sequence
  Description of Artificial Sequence Synthetic primer
ttgactggagttcagacgtgtgctcttccgatctaaaatgacaaagaacagctcaaagc

SEQ ID NO: 128 - DNA, 57 bp, Artificial Sequence
  Description of Artificial Sequence Synthetic primer
ttgactggagttcagacgtgtgctcttccgatctagcacttacctgtgactccatag

SEQ ID NO: 129 - DNA, 56 bp, Artificial Sequence
  Description of Artificial Sequence Synthetic primer
ttgactggagttcagacgtgtgctcttccgatctagatcatctgctggccgtgtgc

SEQ ID NO: 130 - DNA, 54 bp, Artificial Sequence
  Description of Artificial Sequence Synthetic primer
ttgactggagttcagacgtgtgctcttccgatcttgcgcaacagctccttccac

SEQ ID NO: 131 - DNA, 60 bp, Artificial Sequence
  Description of Artificial Sequence Synthetic primer
ttgactggagttcagacgtgtgctcttccgatcttgggcatcactgtaaaccttgcagac

SEQ ID NO: 132 - DNA, 55 bp, Artificial Sequence
  Description of Artificial Sequence Synthetic primer
ttgactggagttcagacgtgtgctcttccgatcttgacagccctctggacaacac

(19) United States
(12) Patent Application Publication
(10) Pub. No.: US 2020/0048692 A1
(43) Pub. Date: Feb. 13, 2020

(54) ENRICHMENT AND DETERMINATION OF NUCLEIC ACIDS TARGETS

(71) Applicant: City University of Hong Kong, Kowloon (HK)

(72) Inventors: Zongli Zheng, Kowloon (HK); Firaol Tamiru Kebede, Kowloon (HK)

(21) Appl. No.: 16/057,004

(22) Filed: Aug. 7, 2018

(51) Int. Cl. C12Q 1/6848

(52) U.S. Cl. CPC C12Q 1/6848

(57) ABSTRACT

Embodiments of the disclosure encompass methods of enriching one or more target nucleic acid sequences utilizing initial linear amplification steps followed by adaptor ligation to the amplified products. In specific embodiments, the linear amplification employs primer extension that spans the target nucleic acid sequences and occurs at least in two or more cycles following which adaptors with unique identifier sequences are attached to the extension products.

### TECHNICAL FIELD

Embodiments of the disclosure include at least the fields of molecular biology, cell biology, nucleic acid amplification, genomics, sequencing, and medical diagnostics.

### BACKGROUND

Genetic abnormalities are the root of many genetic diseases, such as cancers and hereditary diseases, for example. These abnormalities can be exploited for diagnosis to guide treatments and clinical and health managements. The present disclosure satisfies a long-felt need in the art by providing methods and compositions that allow for improved enrichment of certain sequences having a higher efficiency and reduced errors in the enrichment, compared to known techniques.

### BRIEF SUMMARY

The present disclosure allows for methods and compositions related to enrichment of particular target sequences within nucleic acids, such as from a sample including a biological sample. In specific embodiments, the initial step(s) of the methods employ linear amplification of target sequences that ensures greater fidelity of the amplified sequences compared to conventional methods.

In specific embodiments, the methods of the disclosure amplify certain target nucleic acid sequence(s) in multiple rounds of a linear amplification manner prior to attaching particular adaptors to the linearly amplified products. The adaptors may comprise a unique identifier sequence.

In particular embodiments of the methods of the disclosure, there are methods for enriching target sequences in nucleic acids from a biological sample that comprise multiple iterations of primer extension of dissociated nucleic acids that produces a polymerized product comprising the target sequence, followed by adaptor ligation to the extended products. This allows for linear amplification having greater fidelity of the products at initial steps of the enrichment process.

Disclosed in certain embodiments herein are methods for enrichment of target nucleic acids for determining genetic alterations in a biological sample. The enriched nucleic acids may be further manipulated, such as sequencing of part or all of the enriched nucleic acids. The production of greater quantities of the enriched nucleic acids allows for their analysis that may then be utilized in determination of a diagnosis, prognosis, appropriate therapy, or a combination thereof, for example.

Embodiments of the disclosure include methods of enriching at least one target sequence from a nucleic acid sample, comprising the steps of: (a) dissociating nucleic acids comprising the target sequence from the sample into single stranded templates, wherein a single stranded template comprises a first strand comprising the target sequence; (b) annealing to the first strand a primer that binds the first strand at a position that is 3′ in relation to the target sequence on the first strand; (c) extending by polymerization the primer over part or all of the full length of the first strand to produce a primer extension product; (d) dissociating the primer extension product from the first strand; (e) repeating steps (a) through (d) one or more times; and (f) ligating to the 3′ end of the primer extension product an adaptor comprising a first end comprising a double stranded region and a second end comprising a single stranded region, wherein the adaptor comprises a known sequence and wherein the ligation occurs with a 5′ recessed end of the second end of the adaptor, thereby producing a plurality of nucleic acids comprising the target sequence and the known sequence. In additional embodiments, wherein step (a) produces single stranded templates comprising a second strand that is complementary to the first strand, and the second strand comprises a complementary sequence to the target sequence, the method further comprising the steps of: (b′) annealing to the second strand a primer that binds the second strand at a position that is 3′ in relation to the target sequence on the second strand; (c′) extending by polymerization the primer over part or all of the full length of the second strand to produce a primer extension product; (d′) dissociating the primer extension product from the second strand; (e′) repeating steps (b′) through (d′) one or more times; and (f′) ligating to the 3′ end of the primer extension product an adaptor comprising a first end comprising a double stranded region and a second end comprising a single stranded region, wherein the ligation occurs with the 5′ end of the second end of the adaptor, wherein the steps (b′), (c′), (d′), (e′), and (f′) occur at substantially the same time as the respective steps of (b), (c), (d), (e), and (f) above, thereby producing a plurality of nucleic acids comprising the complementary sequence to the target sequence. In some embodiments, the method occurs in a multiplexing manner.

In some embodiments, the method further comprises the step of subjecting the nucleic acids from the sample to dephosphorylation. The methods of the disclosure may also further comprising the step of analyzing one or more plurality of nucleic acids comprising the target sequence and/or further comprising the step of analyzing one or more plurality of nucleic acids comprising the complementary sequence to the target sequence. Adaptors may also comprises a random unique sequence.

The nucleic acid sample may be from a mammal, such as a human, cat, dog, horse, and so forth. The mammal may be known to have or suspected of having a disease, such as cancer. The mammal may be suspected of having a genetic disorder, and the mammal may be a fetus. In cases wherein the individual has or is suspected of having cancer, the one or more target sequences may comprise one or more markers for the cancer. The nucleic acid sample may be from a CRISPR gene editing sample.

In specific cases, the method enriches 2, 3, 4, 5, 6, 7, 8, 9, 10, or more target sequences, including simultaneously enriching 2, 3, 4, 5, 6, 7, 8, 9, 10, or more target sequences.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawing.

FIG. 1 illustrates one example of a method of the disclosure in which (a) double stranded nucleic acids are dissociated to produce first and second strands in which the first strand comprises the target sequence (*); (b) primer extension initiates on the first strand or the second strand, independently; (c) extension occurs to the end of the fragment (at least in some embodiments); (d) the extended product dissociates from the first strand or the second strand, respectively; (d) the steps of (a) through (d) occur two or more times; and (f) particular adaptors are ligated to the linearly amplified extension products.

FIG. 2A demonstrates that upon using 10 ng of Tur-Q7 reference sample (HorizonDx, 1.3% Tier MAF), with the library construction methods disclosed herein and a unique molecular identifier (UMI)-aware variant caller named Bin-based Genotyping (BinGo) for SNV/Indel/di-nucleotide (phase), all expected mutations (100% sensitivity) and no any other mutations (100% specificity) were detected. FIG. 2B shows a graphic presentation of FIG. 2A.

FIG. 3 shows one example of enrichment of a low level variant (MAF, 3.59% [=6772/188979]) for a 15-base deletion in the exon 19 of EGFR (top), and an ultra-low level variant (MAF, 0.05% [=103/187821]) in a second sample (bottom).

### DETAILED DESCRIPTION

As used herein the specification, “a” or “an” may mean one or more. As used herein in the claim(s), when used in conjunction with the word “comprising,” the words “a” or “an” may mean one or more than one. As used herein “another” may mean at least a second or more. In specific embodiments, aspects of the subject matter may “consist essentially of” or “consist of” one or more elements or steps of the subject matter, for example. Some embodiments of the subject matter may consist of or consist essentially of one or more elements, method steps, and/or methods of the subject matter. It is contemplated that any method or composition described herein can be implemented with respect to any other method or composition described herein.

### I. METHOD EMBODIMENTS

The present disclosure provides methods for enrichment and determination of nucleic acids targets that are advantageous over other methods in the art.

In general embodiments of the disclosure, certain nucleic acids comprising target sequence(s) of interest are enriched by increasing their number using primer extension methods in a linear amplification manner prior to affixing adaptors to the amplified products. The methods lack amplification by polymerase chain reaction (PCR) at least at the initial steps of the method. In specific cases, the adaptors are ligated to primer extension products only after multiple cycles of extension and dissociation have occurred, thereby producing linear amplification products. Such steps considerably increase the efficiency of target enrichment by the linear amplification in the present methods, because linear amplification enriches as many original template targets as possible with repeated attempts that maximizes the chance of all of the original targets being contacted and copied into second generation products, and lacks the tendency for copying already copied, i.e. second, third and so forth generations of products that are much more abundant than the original templates associated with non-linear, exponential amplification methods such as PCR. In specific embodiments a target sequence is copied from an original template, whereas other methods enrich targets based on copying of a copied version of the template. The linear amplification methods of the present disclosure keeps to a minimum the production of products with mistakes prior to tagging of the amplified products with adaptors. Such exceptional high efficiency and accuracy is particularly useful for applications that utilize samples in low abundance, although the method may be utilized for samples having abundant nucleic acids as well.

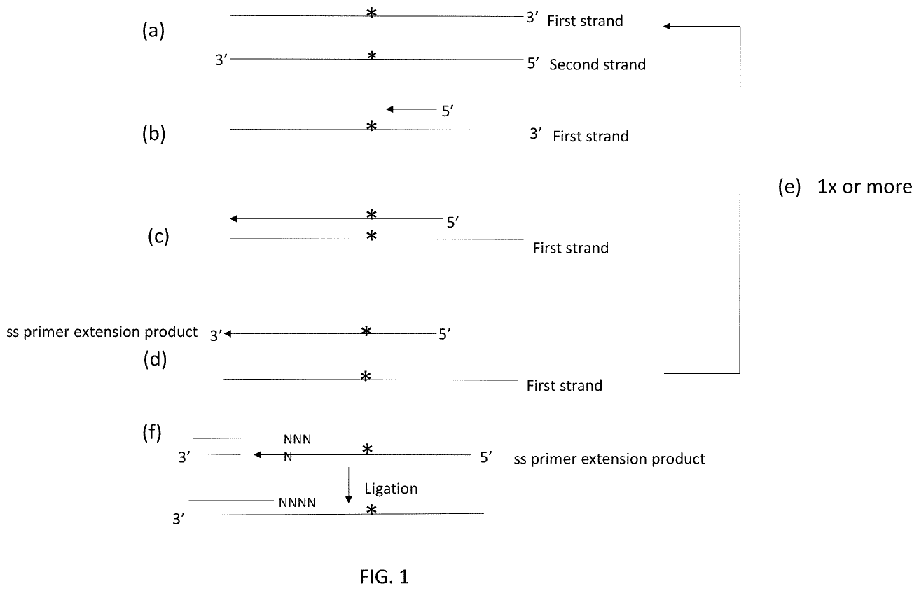

In particular embodiments, there is a method of enriching at least one target sequence from a nucleic acid sample that comprises the following steps: (a) dissociating nucleic acids comprising the target sequence from the sample into single stranded templates, wherein a single stranded template comprises a first strand comprising the target sequence and a second strand comprising the target complementary sequence; (b) annealing to the first strand a primer that binds the first strand at a position that is 3′ in relation to the target sequence on the first strand, and/or to the second strand a primer that binds the second strand at a position that is 3′ in relation to the target sequence on the second strand; (c) extending by polymerization the primer over part or all of the full length of the first and/or the second strands to produce primer extension product; (d) repeating steps (a) through (c) one or more times; and (e) ligating to the 3′ end of the primer extension product an adaptor comprising a double stranded region and a single stranded region, wherein the ligation occurs at a 5′ recessed end of the adaptor, thereby producing a plurality of nucleic acids comprising the target sequence and a known sequence from the adaptor. In FIG. 1, the method is illustrated in which case (d) involving dissociation of the primer extended product from the template is shown. Such methods inherently generate primer extension products in a linearly amplified method that are then ligated (also may be referred to as tagged) with the adaptor. The adaptors may or may not comprise a known sequence (which may be referred to as a unique identifier sequence).

Certain embodiments of the disclosure include methods of enriching one or more target nucleotide sequences, such as target sequence(s) flanking a locus of interest, from a nucleic acid sample comprising nucleic acid templates. Examples of method steps include dissociating double-stranded and/or single-stranded nucleic acid templates comprising the locus of interest into single-stranded templates; annealing an outside target-specific primer to the first strand in the vicinity of the target nucleotide sequence; extending the outside target-specific primer over the full lengths of the first strand using a DNA polymerase to provide a nascent primer extension duplex; dissociating the nascent primer extension duplex at a sufficiently high temperature into the first strand and a single-stranded primer extension product; and then repeating the aforementioned steps for one or more primer extension cycles. Following this, the enriched extension products comprising the locus of interest flanking the target sequence may be ligated to a universal adaptor, for example wherein the universal adaptor is an oligonucleotide comprising a duplex portion and a non-duplex portion, and wherein each nucleic acid extension product is ligated to the universal adaptor at a 5′ recessed end; and wherein each nucleic acid extension product's 3′ end is ligated to the universal adaptor's 5′ end.

Thus, in the present disclosure the methods beneficially have increased chances of accurately identifying nucleic acid targets from a sample because of use of more hybridization/extension cycles prior to adaptor ligation. This is attributed to adaptor ligation only after there is linear amplification of original template(s), and this adaptor ligation occurs in a second generation of extension products such that the amplification utilizes duplicating a sequence of interest from an original nucleic acid instead of duplicating a sequence of interest from a duplicated nucleic acid that may have errors in the sequence. An error may be the wrong nucleotide at a certain position and/or loss or gain of one or more nucleotides at a certain position, for example. Therefore, although methods that utilize conventional PCR amplification prior to tagging of the nucleic acids with adaptors would propagate errors during exponential cycles, the present disclosed methods instead maintain errors at minimal levels because the amplification is non-linear that would suppress the chances of propagating errors. Such methods are particular useful in cases wherein there is a low abundance of biological sample and/or a low abundance of nucleic acids within a biological sample.

Particular aspects of the methods of the disclosure allow for highly multiplex target enrichment. Such enrichment is particularly useful in applications where multiple targets in a sample are in need of being analyzed simultaneously, e.g., in the analysis of multiple gene targets from any kind of sample, including an individual suspected of having cancer or that has cancer. Employing analysis of multiple gene targets using methods of the disclosure increases the accuracy of analysis of samples for diagnosis and/or prognosis and/or therapy monitoring and/or disease monitoring for an individual, for example. In such multiplex methods, multiple target sequences are simultaneously enriched based upon selection of a plurality of appropriate, non-identical primers that hybridize adjacent to the multiple, respective target sequences. In specific embodiments, a primer comprises a common sequence at the 5′ end and a gene target specific primer at the 3′ end. The 5′ end common sequence is of sufficient length (e.g. at least 15 nucleotides long) that, upon primer-primer interaction, forms intramolecular complementary sequences that suppresses amplification of primer dimer during the thermocycling of the reaction, thereby enabling high level of target multiplexing enrichment.

In some embodiments, a target sequence is enriched in addition to a complementary sequence to the target sequence also being enriched, including simultaneously at least in some cases. Such bi-template target enrichment may or may not be included in multiplex methods. In such bi-template target enrichment cases, upon dissociation of the nucleic acids to produce single stranded templates comprising first and second strands that are complementary, the second strand comprises a complementary sequence to the target sequence. In such cases, the method further comprises, simultaneously to the enrichment of the first strand in the same reaction, annealing to the second strand a primer that binds the second strand at a position that is 3′ in relation to the target sequence on the second strand; extending by polymerization the primer over part or all of the full length of the second strand to produce a primer extension product; dissociating the primer extension product from the second strand; and repeating these steps one or more times. Following this, adaptors may be ligated to the extended products corresponding to the second strand. In particular cases, bi-template linear amplification allows for enrichment of both strands of DNA targets, allowing double confirmation of the sequence by filtering away inconsistent downstream sequence observation from the two strands, thereby reduces any sequencing errors introduced starting from template damage until final sequencing.

In certain embodiments, the nucleic acids from the sample may be manipulated in any manner prior to subjecting to the methods of the disclosure. The manipulation may be standard isolation, purification, and/or chemically and/or physically modifying the nucleic acids, for example. In some cases, the nucleic acids may be subjected to a de-phosphorylation step. In addition, or alternatively, the nucleic acids may be digested with an enzyme to produce fragments. In cases wherein there is a de-phosphorylation step of input DNA, this disables non-target DNA to non-target DNA ligation in the subsequent ligation step. Such a modification decreases chimeric DNA artifacts, leading to increased specificity.

The methods of the disclosure utilize multiple rounds of enriching target nucleic acids with primer extension prior to ligation, including at least in some cases 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more rounds of such enriching steps prior to ligation. However, in some embodiments only a certain number of rounds may be utilized prior to ligation. That is, in specific aspects, no more than 2, 3, 4, 5, 6, 7, 8, 9, or 10 primer extension steps are employed prior to ligation.

The nucleic acids that comprise the target sequences may or may not be part of a genome, including a genome of a higher organism. In specific embodiments, the nucleic acids are from a sample from a higher organism, such as a mammal, including a human, dog, cat, horse, cow, and so forth. The mammal may be in need of medical care or may be suspected to be in need of medical care, although the mammal may be in need of routine or preventative care. A nucleic acid sample may be taken from a mammal and may be of any kind, including blood, plasma, hair, cheek scrapings, urine, feces, biopsy (liquid or tissue and may be guided from any device, such as ultrasound or CT scan, for example), cerebrospinal fluid, nipple aspirate, saliva, sputum, mucus, or a combination thereof. The nucleic acids from the samples may be processed by known methods in the art. The samples may be frozen, from storage, or fresh prior to obtaining the nucleic acids therefrom.

In some embodiments, after suitable rounds of linear amplification followed by adaptor ligation, the adaptor-ligated extension products may be further manipulated or modified. For example, they may be sequenced by any manner, including at least next generation sequencing and third/single-molecule sequencing; they may be amplified, such as by polymerase chain reaction; they may be subjected to hybridization; and so forth.

### II. EXAMPLES OF APPLICATIONS OF THE METHODS

The present methods achieve high efficiency for enriching target nucleic acids from a population of nucleic acids with high efficiency and sensitivity when compared with conventional methods, thereby allowing broader applications and enabling circumstances where materials harboring genetic abnormality is scarce. In other cases, the nucleic acids are not scarce in a sample, or the sample is not scarce.

Examples of applications for the methods of enriching target nucleic acid sequences include the trace amount of circulating cell-free tumor DNA (also known as liquid biopsy); circulating tumor DNA (ctDNA) shed from early stage of tumor or from early relapsed of tumor; or the minimal amount of fetal DNA shed into a mother's blood. The methods are capable of enriching highly degraded samples, including ctDNA and formalin-fixed material that are highly fragmented, for example. The methods include production of suitable material for subsequent analysis, such as for genotyping tumors prior to targeted therapy and/or immune-therapy; for early detection of cancers; for non-invasive pre-natal diagnosis; for forensic genotyping; for pharmacogenetics typing; for human leukocyte antigen genotyping; for T-cell receptor repertoire profiling of body immune status; for detection of CRISPR target sites; and/or for evaluation of CRISPR gene editing safety, for example.

The present disclosed methods have a high sensitivity for detection of samples with mutations at low minor allele frequency. The methods have the ability to enrich targets by knowing only one end of certain sequences. The methods can detect gene fusions, structure variation, virus and/or insert DNA integrations into a host genome, one or more SNPs, and so forth.

In specific cases, an individual in need of diagnosis, treatment, prognosis, therapy determination, and/or therapy monitoring may provide an appropriate biological sample. The individual may have or be suspected to have a medical condition having one or more certain sequences associated with the medical condition or that are known to be causative for the medical condition. The sample may be obtained by an individual and the nucleic acids may or may not be immediately processed therefrom. In some cases the nucleic acids are obtained from the sample substantially immediately thereafter, whereas in other cases the nucleic acids are not obtained from the sample until later. The sample and/or nucleic acids may be stored under appropriate conditions for an appropriate time. The individual(s) that obtains the sample may or may not be the individual(s) that later performs the method and/or that analyzes the sequence. Once the nucleic acid is obtained from the sample (for example, by routine steps), the methods of the disclosure are performed on the nucleic acids (that may or may not include the individual's genome as a starting plurality of nucleic acids). Following the enrichment methods of the disclosure, the target sequence(s) from the nucleic acids from the sample are appropriately analyzed. Such analysis provides the intended information regarding diagnosis, prognosis, treatment regimen, therapy monitoring, and so forth.

The target sequence(s) may or may not be associated with a medical condition, such as the sequence having one or more components that are indicative of the presence and/or risk for a certain medical condition and/or susceptibility to a specific treatment for a particular medical condition. The target sequence may comprise one or more particular nucleotides that are associated with a medical condition, and such nucleotide(s) associated with the medical condition may be mutated compared to the same sequence in a healthy individual, or the target sequence may comprise an insertion and/or deletion compared to the same sequence in a healthy individual. In specific cases, the target sequence comprises one or more SNPs that are associated with a medical condition. Such SNPs may or may not be disease-causing SNPs. In any case, there are publically available databases that contain genome variation information, including SNPs, such as at National Center for Biotechnology Information's ClinVar database or European Bioinformatics Institute EBI.

In certain embodiments, the DNA is bisulphite-converted prior to the method. Thus, bisulphite-converted samples may be utilized to enrich for target(s) that possess methylation nucleotide status with high efficiency and accuracy.

Target sequences may be of any suitable length and the length may depend on the following characteristics: nature of the particular sequence within to be analyzed; the percentage of G-C content in the region; the uniqueness of the target sequence itself; and/or the uniqueness of adjacent regions in which a primer could bind. In specific embodiments, the target sequence is at least 20, 21, 22, 23, 24, 25, 26, 27, 28, 29, 30, 35, 40, 45, 50, 100, 150, 200, 250, 300, 350, 400, 450, 500, or more nucleotides in length. However, within the entirety of the target sequence the particular sequence of interest to be analyzed for a mutation, for example, may be only 1, 2, 3, 4, 5, or more nucleotides, for example. In a particular case, a target sequence of 50-300, 50-250, 50-200, 50-150, 50-100, 50-75, 100-300, 100-150, 100-200, 100-150, 150-300, 150-250, 150-200, 150-175, 200-300, 200-250, 250-300, 20-500, 20-450, 20-400, 20-350, 20-300, 20-250, 20-200, 20-150, 20-100, 20-50, 50-500, 50-400, 50-350, 50-300, 50-250, 50-200, 50-150, 50-100, 100-500, 100-400, 100-300, 100-200, 200-500, 200-400, 200-300, 300-500, 300-400, or 300-350 nucleotides may be enriched, but the nature of only 1 nucleotide, 2 nucleotides, or a few nucleotides within the target sequence is of concern.

### III. COMPOSITION EMBODIMENTS

Compositions of the disclosure include at least primers suitable for primer extension and/or adaptors suitable for adaptor ligation.

Primers of the disclosure may be utilized to generate primer extension products that are capable of hybridizing outside of the nucleic acid target sequence such that when the primer is extended, polymerization occurs across the nucleic acid target sequence and the primer extension product then comprises the nucleic acid target sequence. The distance between which the primer binds outside of the nucleic acid target sequence and the nucleic acid target sequence itself may or may not be considered in the design of the primer (and therefore the sequence to which the primer binds). However, in specific cases, the primer binds within 1-300, 1-200, 1-100, 1-50, 25-300, 25-250, 25-200, 25-100, 25-50, 50-300, 50-250, 50-200, 50-100, 100-300, 100-200, 200-300, or 250-300 nucleotides of the target nucleic acid sequence. The primer may be of any suitable sequence so long as it is capable of hybridizing to the template to initiate extension. Therefore, across the totality of the primer sequence the hybridization may be complete or there may be one or a few nucleotides that do not have an exact hybridization match between the primer and template. The primer preferably has an exact match to its template counterpart sequence at the 3′ end of the primer. The extent of mismatches may be dictated by the size of the primer, and this can be routinely determined by the skilled artisan. In specific embodiments, the primer is of a particular length, including at least 18 nucleotides but no more than 100 nucleotides. The primer may or may not have 40-60% G-C content.

In particular embodiments, an adaptor has a region that is double stranded and a region that is single stranded. In specific aspects, the adaptor comprises a duplex portion at one end (“a first end”) and a non-duplex portion at the other end (“a second end”). In specific embodiments, the adaptor is configured such that a nucleic acid extension product is capable of being ligated to the adaptor through the 5′ recessed end. In specific cases, the ligation occurs at the 3′ end of the nucleic acid extension product.

In particular cases, the adaptor comprises two strands, each of which may or may not be of a certain length. A shorter strand may range from 6-50, 6-40, 6-30, 6-20, 6-15, 10-50, 10-40, 10-30, 10-20, 20-50, 20-40, 20-30, 30-50, 30-40, or 40-50 nucleotides in length, and the longer strand may range from 15-100 nucleotides. The end of the adaptor that is single stranded may be of any suitable length, including in a range of between 4-20, 4-17, 4-15, 4-10, 4-7, 5-20, 5-15, 5-10, 8-20, 8-15, 8-12, 10-20, 10-15, 10-12, 12-20, 12-18, 12-15, 15-20, 15-17, or 17-20 nucleotides. The double stranded region of the adaptor may be in a range from 8-100 nucleotides in length, for example, including 8-100, 10-100, 20-100, 30-100, 40-100, 50-100, 60-100, 70-100, 80-100, 90-100, 8-90, 8-80, 8-70, 8-60, 8-50, 8-40, 8-30, 8-20, 8-15, 8-12, 12-50, 12-50, 12-30, 12-25, 12-20, 12-18, 15-40, 15-35, 15-30, 15-20, 20-90, 20-80, 20-70, 20-60, 20-50, 20-40, 20-30, 30-90, 30-80, 30-70, 30-60, 30-50, 30-40, 40-90, 40-80, 40-70, 40-60, 40-50, 50-90, 50-80, 50-70, 50-60, 60-90, 60-80, 60-70, 70-90, 70-80, 80-20, and so forth. The adaptor may or may not comprise a particular unique identifier sequence. The unique identifier sequence may be of any suitable sequence so long as nucleic acids that comprise the unique sequence may be able to be utilized as a barcode, as a molecule identifier, and/or may be targeted subsequently for amplification via binding of oligonucleotides to at least part of the unique sequence. The unique identifier sequence may be at least 2, 3, 4, 5, 6, 7, 8, 9, or 10 nucleotides in length but may be no more than 20, 19, 18, 17, 16, 15, 14, 13, 12, or 11 nucleotides in length.

In specific embodiments, the adaptors comprise a label, for example a radionuclide, an affinity tag, a hapten, an enzyme, a chromophore, or a fluorophore.

In some embodiments, the adaptor comprises a phosphor at the 5′ recessed end of the oligonucleotide for ligation to the 3′ end of a single strand extension product. The common sequence within the adaptor may or may not be the sequences used in next-generation sequencing platforms.

### EXAMPLE

The following example is included to demonstrate particular embodiments of the disclosure. It should be appreciated by those of skill in the art that the techniques disclosed in the example that follows represent techniques discovered by the inventor to function well in the practice of the subject matter of the disclosure, and thus can be considered to constitute particular modes for its practice. However, those of skill in the art should, in light of the present disclosure, appreciate that many changes can be made in the specific embodiments that are disclosed and still obtain a like or similar result without departing from the spirit and scope of the invention.

### Example 1

### Enrichment and Determination of Nucleic Acids Targets and Examples of Compositions

Sensitivity and specificity of mutation detection was demonstrated using reference standard materials with examples of known mutations in one sample. The results showed 100% sensitivity and 100% specificity for detection of all of the 44 variants of different types and at 1% minor allele frequency (MAF) in one single sample, using 10 ng. Other amounts of starting samples may be utilized, including smaller or greater. The range of starting material may be at least 1 ng or less, or may be 100 pg or less. The starting material may be at least 1 pg, at least 10 pg, at least 100 pg, at least 1 ng, and so forth.

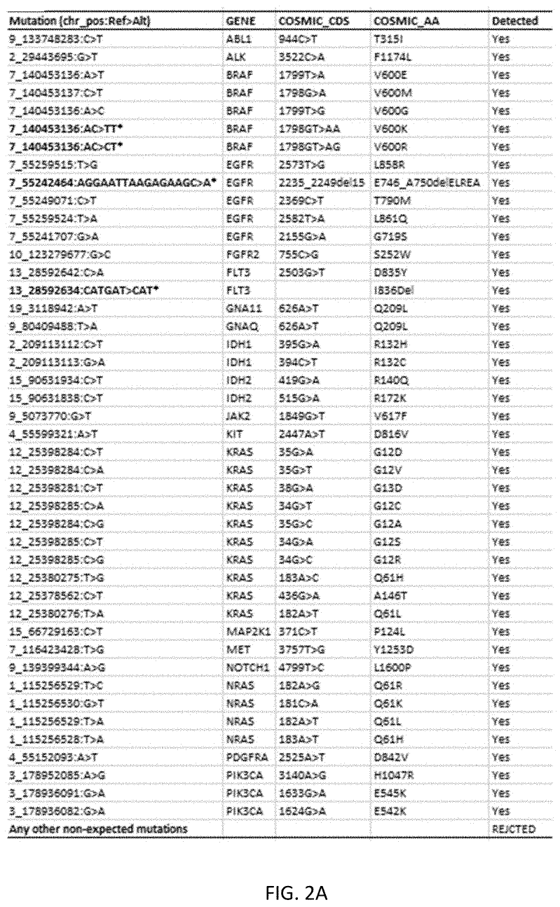

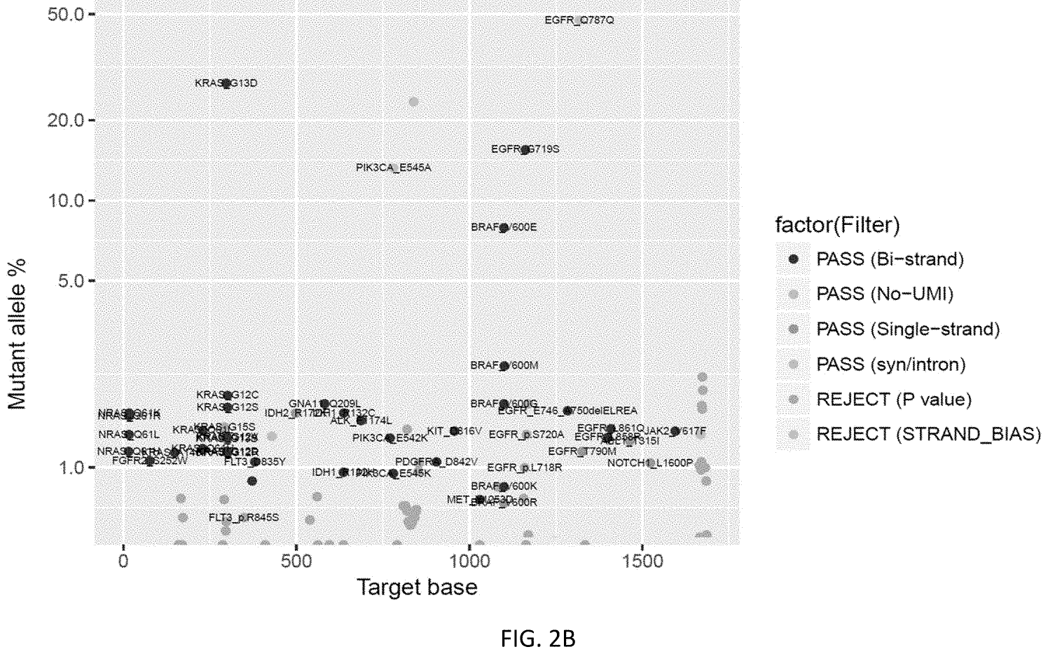

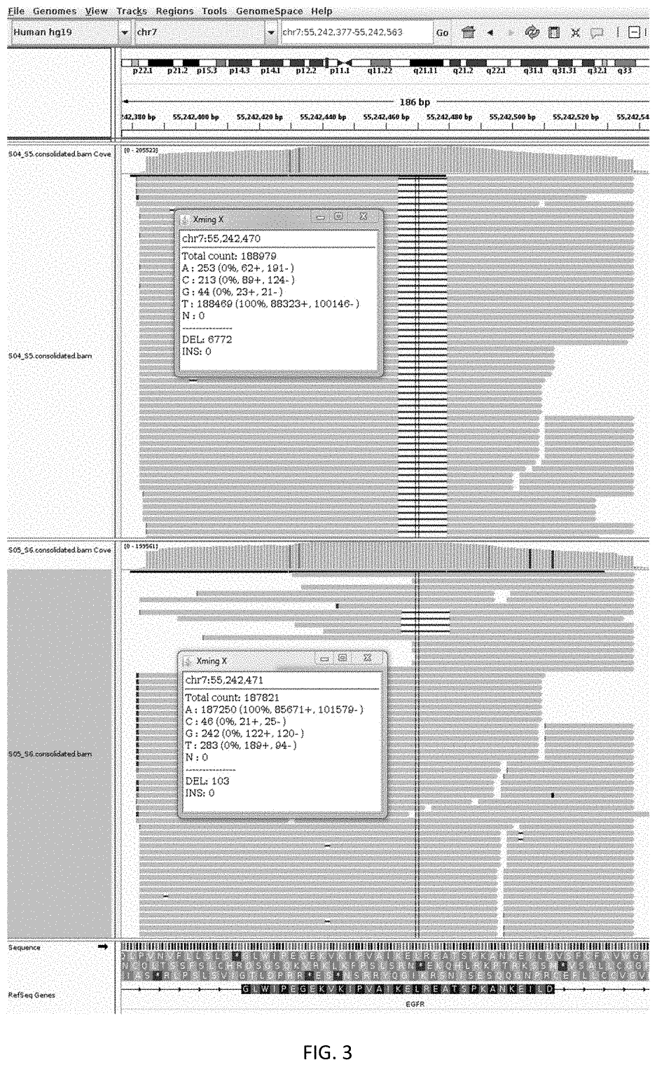

In particular, FIGS. 2A and 2B provide evidence of the 44 variants at 1% minor allele frequency (MAF) and filtered noises enabled by the bi-strand and unique molecular identifier. FIG. 3 shows one embodiment of enrichment of a low level variant (MAF, 0.05%) for a deletion in the EGFR, as an example of a mutation.

For illustrative purposes only, Tables 1, 2, and 3 provide examples of adaptor sequences, gene target specific primer 1 sequences, and gene target specific primer 2 sequences, respectively.

TABLE 1
Adaptor sequences

| Name | sequence | SEQ ID NO |
|---|---|---|
| LS_T001 | AACUACACGACGCUCUUCCGAUCUNNWNNWNATGCCATGCCGTNNNNNNNN | 1 |
| LS_T002 | AACUACACGACGCUCUUCCGAUCUNNWNNWNACCATCCAACGANNNNNNNN | 2 |
| LS_T003 | AACUACACGACGCUCUUCCGAUCUNNWNNWNGTGGCCTACTACNNNNNNNN | 3 |
| LS_T004 | AACUACACGACGCUCUUCCGAUCUNNWNNWNGGAGTTGAGGTGNNNNNNNN | 4 |
| LS_T005 | AACUACACGACGCUCUUCCGAUCUNNWNNWNATTGCAAGCAACNNNNNNNN | 5 |
| LS_T006 | AACUACACGACGCUCUUCCGAUCUNNWNNWNTACAACCGAGTANNNNNNNN | 6 |
| LS_T007 | AACUACACGACGCUCUUCCGAUCUNNWNNWNTGCGTTCTAGCGNNNNNNNN | 7 |
| LS_T008 | AACUACACGACGCUCUUCCGAUCUNNWNNWNTGTTCCTCTCACNNNNNNNN | 8 |
| LS_T009 | AACUACACGACGCUCUUCCGAUCUNNWNNWNTGCAGTCCTCGANNNNNNNN | 9 |
| LS_T010 | AACUACACGACGCUCUUCCGAUCUNNWNNWNCGATACACTGCCNNNNNNNN | 10 |
| LS_T011 | AACUACACGACGCUCUUCCGAUCUNNWNNWNTCTGCGAGTCTGNNNNNNNN | 11 |
| LS_T012 | AACUACACGACGCUCUUCCGAUCUNNWNNWNTCTTGCGGAGTCNNNNNNNN | 12 |
| LS_T013 | AACUACACGACGCUCUUCCGAUCUNNWNNWNAGGCTTACGTGTNNNNNNNN | 13 |
| LS_T014 | AACUACACGACGCUCUUCCGAUCUNNWNNWNTCACGAGTCACANNNNNNNN | 14 |
| LS_T015 | AACUACACGACGCUCUUCCGAUCUNNWNNWNAGCCGGAGAGTANNNNNNNN | 15 |
| LS_T016 | AACUACACGACGCUCUUCCGAUCUNNWNNWNATGGAAGGTGGCNNNNNNNN | 16 |
| LS_B001 | /5Phos/ACGGCATGGCATNWNNWNNAGATCGGAAGAGCGTCGTGTAG | 17 |
| LS_B002 | /5Phos/TCGTTGGATGGTNWNNWNNAGATCGGAAGAGCGTCGTGTAG | 18 |
| LS_B003 | /5Phos/GTAGTAGGCCACNWNNWNNAGATCGGAAGAGCGTCGTGTAG | 19 |
| LS_B004 | /5Phos/CACCTCAACTCCNWNNWNNAGATCGGAAGAGCGTCGTGTAG | 20 |
| LS_B005 | /5Phos/GTTGCTTGCAATNWNNWNNAGATCGGAAGAGCGTCGTGTAG | 21 |
| LS_B006 | /5Phos/TACTCGGTTGTANWNNWNNAGATCGGAAGAGCGTCGTGTAG | 22 |
| LS_B007 | /5Phos/CGCTAGAACGCANWNNWNNAGATCGGAAGAGCGTCGTGTAG | 23 |
| LS_B008 | /5Phos/GTGAGAGGAACANWNNWNNAGATCGGAAGAGCGTCGTGTAG | 24 |
| LS_B009 | /5Phos/TCGAGGACTGCANWNNWNNAGATCGGAAGAGCGTCGTGTAG | 25 |
| LS_B010 | /5Phos/GGCAGTGTATCGNWNNWNNAGATCGGAAGAGCGTCGTGTAG | 26 |
| LS_B011 | /5Phos/CAGACTCGCAGANWNNWNNAGATCGGAAGAGCGTCGTGTAG | 27 |
| LS_B012 | /5Phos/GACTCCGCAAGANWNNWNNAGATCGGAAGAGCGTCGTGTAG | 28 |
| LS_B013 | /5Phos/ACACGTAAGCCTNWNNWNNAGATCGGAAGAGCGTCGTGTAG | 29 |
| LS_B014 | /5Phos/TGTGACTCGTGANWNNWNNAGATCGGAAGAGCGTCGTGTAG | 30 |
| LS_B015 | /5Phos/TACTCTCCGGCTNWNNWNNAGATCGGAAGAGCGTCGTGTAG | 31 |
| LS_B016 | /5Phos/GCCACCTTCCATNWNNWNNAGATCGGAAGAGCGTCGTGTAG | 32 |

TABLE 2
Gene Target Specific Primer 1(GSP1)

| | | SEQ |
|---|---|---|
| | | ID |
| Name | Seq | NO |
| KIT.D816_53am1 | GGATCTCGACGCTCTCCCTCTCCTTACTCATGGTCGGAT*C | 33 |
| KIT.D816_53ap1 | GGATCTCGACGCTCTCCCTTCCTTTGCAGGACTGTCAAGC*A | 34 |
| MET.Y1253_32am1 | GGATCTCGACGCTCTCCCTGTCCTTTCTGTAGGCTGGAT*G | 35 |
| MET.Y1253_32ap1 | GGATCTCGACGCTCTCCCTTTTTGAGTTTGCAGACTTTC*C | 36 |
| GNA11.Q209_32am1 | GGATCTCGACGCTCTCCCTGGATTGCAGATTGGGCCTTGG*G | 37 |
| GNA11.Q209_32ap1 | GGATCTCGACGCTCTCCCTGGATGTCACGTTCTCAAAG*C | 38 |
| GNAQ.Q209_32am1 | GGATCTCGACGCTCTCCCTAGCGCTACTAGAAACATGATA*G | 39 |
| GNAQ.Q209_32ap1 | GGATCTCGACGCTCTCCCTTGTTGACTTTTTAGTTTTACTT*T | 40 |
| IDH2.R140R172_107am1 | GGATCTCGACGCTCTCCCTAGGTCAGTGGATCCCCTCTC*C | 41 |
| IDH2.R140R172_107ap1 | GGATCTCGACGCTCTCCCTTGAAGAAGATGTGGAAAAGTC*C | 42 |
| NRAS.Q61_28am1 | GGATCTCGACGCTCTCCCTCTGTCCTCATGTATTGGTCT*C | 43 |
| NRAS.Q61_28ap1 | GGATCTCGACGCTCTCCCTCTGTCCTCATGTATTGGTCT*C | 44 |
| MAP2K1.P124_22am1 | GGATCTCGACGCTCTCCCTCAGAAAACAAGTGGTTATAGATG*G | 45 |
| MAP2K1.P124_22ap1 | GGATCTCGACGCTCTCCCTGCTCCATGCAGATACTGATC*T | 46 |
| FLT3.D835ITD_122am1 | GGATCTCGACGCTCTCCCTGACACAACACAAAATAGCC*G | 47 |
| FLT3.D835ITD_122ap1 | GGATCTCGACGCTCTCCCTACTCCAGGATAATACACATCA*C | 48 |
| ALK.F1174_27am1 | GGATCTCGACGCTCTCCCTTGAGGCTCACCCCAATGCA*G | 49 |
| ALK.F1174_27ap1 | GGATCTCGACGCTCTCCCTAGCTGCTGAAAATGTAACTTTGTAT*C | 50 |
| BRAF.V600_42am1 | GGATCTCGACGCTCTCCCTCAAAATGGATCCAGACAACT*G | 51 |
| BRAF.V600_42ap1 | GGATCTCGACGCTCTCCCTGCTTGCTCTGATAGGAAAATGA*G | 52 |
| JAK2.V617_22am1 | GGATCTCGACGCTCTCCCTAGCAAGTATGATGAGCAAG*C | 53 |
| JAK2.V617_22ap1 | GGATCTCGACGCTCTCCCTATGCTCTGAGAAAGGCATTA*G | 54 |
| KRAS.A146_42am1 | GGATCTCGACGCTCTCCCTGATTTTGCAGAAAACAGATC*T | 55 |
| KRAS.A146_42ap1 | GGATCTCGACGCTCTCCCTGCCTTCTAGAACAGTAGACA*C | 56 |
| KRAS.G12G13_25am1 | GGATCTCGACGCTCTCCCTAAATGATTCTGAATTAGCTGTATC*G | 57 |
| KRAS.G12G13_25ap1 | GGATCTCGACGCTCTCCCTTTTATTATAAGGCCTGCTGAAAAT*G | 58 |
| KRAS.Q61_42am1 | GGATCTCGACGCTCTCCCTAAAGAAAGCCCTCCCCAGT*C | 59 |
| KRAS.Q61_42ap1 | GGATCTCGACGCTCTCCCTCCTACAGGAAGCAAGTAGTAATT*G | 60 |
| ABL1.T315_32am1 | GGATCTCGACGCTCTCCCTGTTGAAGTCCTCGTTGTCTT*G | 61 |
| ABL1.T315_32ap1 | GGATCTCGACGCTCTCCCTTTGCACTCCCTCAGGTAGT*C | 62 |
| EGFR.Ex19_101am1 | GGATCTCGACGCTCTCCCTCAGTTAACGTCTTCCTTCTCT*C | 63 |
| EGFR.Ex19_101ap1 | GGATCTCGACGCTCTCCCTGGACCCCCACACAGCAAAG*C | 64 |
| EGFR.G719_14am1 | GGATCTCGACGCTCTCCCTTTACACCCAGTGGAGAAGCT*C | 65 |
| EGFR.G719_14ap1 | GGATCTCGACGCTCTCCCTCCCACCAGACCATGAGAGG*C | 66 |
| EGFR.L858L861_38am1 | GGATCTCGACGCTCTCCCTAACGTACTGGTGAAAACAC*C | 67 |
| EGFR.L858L861_38ap1 | GGATCTCGACGCTCTCCCTCAGGAAAATGCTGGCTGAC*C | 68 |
| EGFR.T790_35am1 | GGATCTCGACGCTCTCCCTGTGGACAACCCCCACGTGTG*C | 69 |
| EGFR.T790_35ap1 | GGATCTCGACGCTCTCCCTCTGGGAGCCAATATTGTCTT*T | 70 |
| IDH1.R132_27am1 | GGATCTCGACGCTCTCCCTACATTATTGCCAACATGACTTA*C | 71 |
| IDH1.R132_27ap1 | GGATCTCGACGCTCTCCCTAATATCCCCCGGCTTGTGA*G | 72 |
| PDGFRA.D842_32am1 | GGATCTCGACGCTCTCCCTGTGTCCACCGTGATCTGGCTGCT*C | 73 |
| PDGFRA.D842_32ap1 | GGATCTCGACGCTCTCCCTTGAGGACGTACACTGCCTTT*C | 74 |
| PIK3CA.E524_48am1 | GGATCTCGACGCTCTCCCTTGACATTGCATACATTCGAAA*G | 75 |
| PIK3CA.E524_48ap1 | GGATCTCGACGCTCTCCCTTTTCAGTTCAATGCATGCT*G | 76 |
| PIK3CA.H1047_45am1 | GGATCTCGACGCTCTCCCTGGAAAATGACAAAGAACAGCT*C | 77 |
| PIK3CA.H1047_45ap1 | GGATCTCGACGCTCTCCCTCCATTTTAGCACTTACCTGT*G | 78 |
| NOTCH1.L1600_32am1 | GGATCTCGACGCTCTCCCTTAGTAGGGGAAGATCATCTG*C | 79 |
| NOTCH1.L1600_32ap1 | GGATCTCGACGCTCTCCCTGCCGGAGCAGCTGCGCAACA*G | 80 |
| FGFR2.S252_10am1 | GGATCTCGACGCTCTCCCGGGCATCACTGTAAACCTT*G | 81 |
| FGFR2.S252_10ap1 | GGATCTCGACGCTCTCCCTTCACTGACAGCCCTCTGGA*C | 82 |

TABLE 3
Gene Target Specific Primer 2(GSP2)

| | | SEQ |
|---|---|---|
| | | ID |
| Name | Seq | NO |
| KIT.D816_53am2 | TTGACTGGAGTTCAGACGTGTGCTCTTCCGATCTTCCTTACTCATGGTCGGATCACAAAG | 83 |
| KIT.D816_53ap2 | TTGACTGGAGTTCAGACGTGTGCTCTTCCGATCTCCTTTGCAGGACTGTCAAGCAGAGAA | 84 |
| MET.Y1253_32am2 | TTGACTGGAGTTCAGACGTGTGCTCTTCCGATCTGTAGGCTGGATGAAAAATTCACAGTC | 85 |
| MET.Y1253_32ap2 | TTGACTGGAGTTCAGACGTGTGCTCTTCCGATCTTTTTGAGTTTGCAGACTTTCCAAAGC | 86 |
| GNA11.Q209_32am2 | TTGACTGGAGTTCAGACGTGTGCTCTTCCGATCTGGCTGAGTCCTGGCGCTGTG | 87 |
| GNA11.Q209_32ap2 | TTGACTGGAGTTCAGACGTGTGCTCTTCCGATCTCGTTCTCAAAGCAGTGGATC | 88 |
| GNAQ.Q209_32am2 | TTGACTGGAGTTCAGACGTGTGCTCTTCCGATCTCTACTAGAAACATGATAGAGGTGAC | 89 |
| GNAQ.Q209_32ap2 | TTGACTGGAGTTCAGACGTGTGCTCTTCCGATCTTGTTATTAATATGAGTATTGTTAACC | 90 |
| IDH2.R140R172_107am2 | TTGACTGGAGTTCAGACGTGTGCTCTTCCGATCTTGGCCTACCTGGTCGCCATG | 91 |
| IDH2.R140R172_107ap2 | TTGACTGGAGTTCAGACGTGTGCTCTTCCGATCTAGATGTGGAAAAGTCCCAATG | 92 |
| NRAS.Q61_28am2 | TTGACTGGAGTTCAGACGTGTGCTCTTCCGATCTCTGTCCTCATGTATTGGTCTCTCATG | 93 |
| NRAS.Q61_28ap2 | TTGACTGGAGTTCAGACGTGTGCTCTTCCGATCTAAAACAAGTGGTTATAGATGGTGAAA | 94 |
| MAP2K1.P124_22am2 | TTGACTGGAGTTCAGACGTGTGCTCTTCCGATCTTTTCTCCAGCTAATTCATCTGG | 95 |
| MAP2K1.P124_22ap2 | TTGACTGGAGTTCAGACGTGTGCTCTTCCGATCTGCCATCGCTGTAGAACGCACCA | 96 |
| FLT3.D835ITD_122am2 | TTGACTGGAGTTCAGACGTGTGCTCTTCCGATCTACACAACACAAAATAGCCGTATAA | 97 |
| FLT3.D835ITD_122ap2 | TTGACTGGAGTTCAGACGTGTGCTCTTCCGATCTCATCACAGTAAATAACACTCTGG | 98 |
| ALK.F1174_27am2 | TTGACTGGAGTTCAGACGTGTGCTCTTCCGATCTCAGGCTCACCCCAATGCAGCGAAC | 99 |
| ALK.F1174_27ap2 | TTGACTGGAGTTCAGACGTGTGCTCTTCCGATCTATGTAACTTTGTATCCTGTTCCTC | 100 |
| BRAF.V600_42am2 | TTGACTGGAGTTCAGACGTGTGCTCTTCCGATCTACAACTGTTCAAACTGATGGG | 101 |
| BRAF.V600_42ap2 | TTGACTGGAGTTCAGACGTGTGCTCTTCCGATCTTGCTCTGATAGGAAAATGAGATCTAC | 102 |
| JAK2.V617_22am2 | TTGACTGGAGTTCAGACGTGTGCTCTTCCGATCTATGATGAGCAAGCTTTCTCAC | 103 |
| JAK2.V617_22ap2 | TTGACTGGAGTTCAGACGTGTGCTCTTCCGATCTGCATTAGAAAGCCTGTAGTTTTAC | 104 |
| KRAS.A146_42am2 | TTGACTGGAGTTCAGACGTGTGCTCTTCCGATCTAACAGATCTGTATTTATTTCAGT | 105 |
| KRAS.A146_42ap2 | TTGACTGGAGTTCAGACGTGTGCTCTTCCGATCTGCCTTCTAGAACAGTAGACACAAAAC | 106 |
| KRAS.G12G13_25am2 | TTGACTGGAGTTCAGACGTGTGCTCTTCCGATCTTCTGAATTAGCTGTATCGTCAAGG | 107 |
| KRAS.G12G13_25ap2 | TTGACTGGAGTTCAGACGTGTGCTCTTCCGATCTGGCCTGCTGAAAATGACTGA | 108 |
| KRAS.Q61_42am2 | TTGACTGGAGTTCAGACGTGTGCTCTTCCGATCTTCCCCAGTCCTCATGTACTG | 109 |
| KRAS.Q61_42ap2 | TTGACTGGAGTTCAGACGTGTGCTCTTCCGATCTAGGAAGCAAGTAGTAATTGATGGAG | 110 |
| ABL1.T315_32am2 | TTGACTGGAGTTCAGACGTGTGCTCTTCCGATCTGTCCTCGTTGTCTTGTTGGC | 111 |
| ABL1.T315_32ap2 | TTGACTGGAGTTCAGACGTGTGCTCTTCCGATCTGCACTCCCTCAGGTAGTCCAGGAG | 112 |
| EGFR.Ex19_101am2 | TTGACTGGAGTTCAGACGTGTGCTCTTCCGATCTGTTAACGTCTTCCTTCTCTCTCTGTC | 113 |
| EGFR.Ex19_101ap2 | TTGACTGGAGTTCAGACGTGTGCTCTTCCGATCTCCCACACAGCAAAGCAGAAAC | 114 |
| EGFR.G719_14am2 | TTGACTGGAGTTCAGACGTGTGCTCTTCCGATCTGGAGAAGCTCCCAACCAAGC | 115 |
| EGFR.G719_14ap2 | TTGACTGGAGTTCAGACGTGTGCTCTTCCGATCTCAGACCATGAGAGGCCCTGCG | 116 |
| EGFR.L858L861_38am2 | TTGACTGGAGTTCAGACGTGTGCTCTTCCGATCTGGTGAAAACACCGCAGCATG | 117 |
| EGFR.L858L861_38ap2 | TTGACTGGAGTTCAGACGTGTGCTCTTCCGATCTGAAAATGCTGGCTGACCTAAAGC | 118 |
| EGFR.T790_35am2 | TTGACTGGAGTTCAGACGTGTGCTCTTCCGATCTCCCACGTGTGCCGCCTGCTG | 119 |
| EGFR.T790_35ap2 | TTGACTGGAGTTCAGACGTGTGCTCTTCCGATCTAGCCAATATTGTCTTTGTGTTC | 120 |
| IDH1.R132_27am2 | TTGACTGGAGTTCAGACGTGTGCTCTTCCGATCTATTGCCAACATGACTTACTTGATCCC | 121 |
| IDH1.R132_27ap2 | TTGACTGGAGTTCAGACGTGTGCTCTTCCGATCTAATATCCCCCGGCTTGTGAGTGGATG | 122 |
| PDGFRA.D842_32am2 | TTGACTGGAGTTCAGACGTGTGCTCTTCCGATCTCGTGATCTGGCTGCTCGCAACG | 123 |
| PDGFRA.D842_32ap2 | TTGACTGGAGTTCAGACGTGTGCTCTTCCGATCTGAGGACGTACACTGCCTTTCGACAC | 124 |
| PIK3CA.E524_48am2 | TTGACTGGAGTTCAGACGTGTGCTCTTCCGATCTAGCCTTAGATAAAACTGAGCAAG | 125 |
| PIK3CA.E524_48ap2 | TTGACTGGAGTTCAGACGTGTGCTCTTCCGATCTCATGCTGTTTAATTGTGTGGAAG | 126 |
| PIK3CA.H1047_45am2 | TTGACTGGAGTTCAGACGTGTGCTCTTCCGATCTAAAATGACAAAGAACAGCTCAAAGC | 127 |
| PIK3CA.H1047_45ap2 | TTGACTGGAGTTCAGACGTGTGCTCTTCCGATCTAGCACTTACCTGTGACTCCATAG | 128 |
| NOTCH1.L1600_32am2 | TTGACTGGAGTTCAGACGTGTGCTCTTCCGATCTAGATCATCTGCTGGCCGTGTGC | 129 |
| NOTCH1.L1600_32ap2 | TTGACTGGAGTTCAGACGTGTGCTCTTCCGATCTTGCGCAACAGCTCCTTCCAC | 130 |
| FGFR2.S252_10am2 | TTGACTGGAGTTCAGACGTGTGCTCTTCCGATCTTGGGCATCACTGTAAACCTTGCAGAC | 131 |
| FGFR2.S252_10ap2 | TTGACTGGAGTTCAGACGTGTGCTCTTCCGATCTTGACAGCCCTCTGGACAACAC | 132 |

### IV. KITS

Any of the compositions described herein may be comprised in a kit. The kits may comprise a suitably aliquoted primer and/or adaptor compositions of the present disclosure, for example. The kit may also include one or more reagents that facilitate practice of the methods, such as enzymes, nucleotides, buffers, and so forth. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The kits of the present invention also will typically include a means for containing any reagent container(s) in close confinement for commercial sale. Such containers may include injection or blow-molded plastic containers into which the desired vials are retained, for example.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method of enriching at least one target sequence from a nucleic acid sample, comprising the steps of:
(a) dissociating nucleic acids comprising the target sequence from the sample into single stranded templates, wherein a single stranded template comprises a first strand comprising the target sequence;
(b) annealing to the first strand a primer that binds the first strand at a position that is 3′ in relation to the target sequence on the first strand;
(c) extending by polymerization the primer over part or all of the full length of the first strand to produce a primer extension product;
(d) dissociating the primer extension product from the first strand;
(e) repeating steps (a) through (d) one or more times; and
(f) ligating to the 3′ end of the primer extension product an adaptor comprising a first end comprising a double stranded region and a second end comprising a single stranded region, wherein the adaptor comprises a known sequence and wherein the ligation occurs with a 5′ recessed end of the second end of the adaptor thereby producing a plurality of nucleic acids comprising the target sequence and the known sequence.

2. The method of claim 2, wherein step (a) produces single stranded templates comprising a second strand that is complementary to the first strand, and said second strand comprises a complementary sequence to the target sequence, said method further comprising the steps of:
(b′) annealing to the second strand a primer that binds the second strand at a position that is 3′ in relation to the target sequence on the second strand;
(c′) extending by polymerization the primer over part or all of the full length of the second strand to produce a primer extension product;
(d′) dissociating the primer extension product from the second strand;
(e′) repeating steps (b′) through (d′) one or more times; and
(f′) ligating to the 3′ end of the primer extension product an adaptor comprising a first end comprising a double stranded region and a second end comprising a single stranded region, wherein the ligation occurs with the 5′ end of the second end of the adaptor,
wherein said steps (b′), (c′), (d′), (e′), and (f′) occur at substantially the same time as the respective steps in claim 1,
thereby producing a plurality of nucleic acids comprising the complementary sequence to the target sequence.

3. The method of claim 1 or 2, further comprising the step of subjecting the nucleic acids from the sample to dephosphorylation.

4. The method of claim 1, further comprising the step of analyzing one or more plurality of nucleic acids comprising the target sequence.

5. The method of claim 1, further comprising the step of analyzing one or more plurality of nucleic acids comprising the complementary sequence to the target sequence.

6. The method of any one of claims 1-5, wherein the adaptor comprises a known unique sequence.

7. The method of any one of claims 1-6, wherein the method occurs in a multiplexing manner.

8. The method of any one of claims 1-7, wherein the method enriches 2, 3, 4, 5, 6, 7, 8, 9, 10, or more target sequences.

9. The method of claim 8, wherein the method simultaneously enriches 2, 3, 4, 5, 6, 7, 8, 9, 10, or more target sequences.

10. The method of any one of claims 1-7, wherein the nucleic acid sample is from a mammal.

11. The method of claim 10, wherein the mammal is a human.

12. The method of claim 11, wherein the human is an individual known to have or suspected of having a disease.

13. The method of claim 12, wherein the disease is cancer.

14. The method of claim 13, wherein the one or more target sequences comprises one or more markers for the cancer.

15. The method of claim 12, wherein the suspected disease is a genetic disorder.

16. The method of claim 15, wherein the individual is a fetus.

17. The method of any one of claims 1-16, wherein the nucleic acid sample is from a CRISPR gene editing sample.
